SEQUENCE LISTING (1 sequence)

SEQ ID NO: 1 - protein, 4 aa, Artificial Sequence
  Synthetic Peptide
PKEK

(19) United States
(12) Patent Application Publication
(10) Pub. No.: US 2012/0244094 A1
(43) Pub. Date: Sep. 27, 2012

(54) TETRAPEPTIDES FOR BRIGHTENING THE SKIN

(75) Inventors: Mike Farwick, Essen (DE); Ursula Maczkiewitz, Essen (DE); Tim Koehler, Dorsten (DE)

(73) Assignee: EVONIK GOLDSCHMIDT GMBH, Essen (DE)

(21) Appl. No.: 13/502,261

(22) PCT Filed: Oct. 18, 2010
(86) PCT No.: PCT/EP10/65588

§ 371 Date: Apr. 16, 2012

(30) Foreign Application Priority Data
Nov. 17, 2009 (DE) 10 2009 046 780.7

(51) Int. Cl. A61K 8/64; A61Q 19/04; A61K 8/67; A61Q 17/02; A61Q 15/00; A61K 8/92; A61Q 19/02; A61Q 17/04

(52) U.S. Cl. 424/60; 424/62; 424/59

(57) ABSTRACT

The invention relates to the use of the tetrapeptide PKEK for brightening human skin, for bleaching pigmented spots and/or for leveling out irregularities in skin coloration.

### FIELD OF THE INVENTION

The present invention relates to the use of the tetrapeptide PKEK for lightening the color of human skin, for bleaching pigment spots and/or for evening out irregularities in skin coloration.

### PRIOR ART

It is the aim of care cosmetics to maintain the impression of an outward youthful appearance, for example that of the skin and hair. In principle, different ways are available for achieving this path. For example, existing skin damage, such as irregular pigmentation or wrinkle formation, can be taken care of with concealing powders or creams. Another approach is to protect the skin against environmental influences which lead to permanent damage and thus aging of the skin. The idea is thus to intervene in a preventative manner and thereby delay the aging process.

The most important function of the skin is to protect the body from the uncontrolled escape of water, on the one hand, and also against the entry of harmful chemicals or bacteria and also of solar radiation, on the other hand. If the human skin is exposed to long-term solar irradiation, this can lead to the appearance of photo-induced skin aging and pigment disorders. This harmful effect of sunlight is attributed inter alia to the UVB radiation (280-320 nm) present in the spectrum of sunlight.

Pigment disorders are perceived as cosmetic flaws. Examples of these local hyperpigmentations would be: freckles, melasma, chloasma, post-inflammatory hyperpigmentation, liver spots and many more. It is common to all forms of hyperpigmentation that a disturbance in the melanogenesis arises.

The pigmentation of the skin is quite essentially determined by the skin's own pigment melanin. This is formed by specialized cells in the epidermis, the melanocytes. During the synthesis of melanin, tyrosinase, being the pacemaker enzyme, plays a very decisive role. The skin reacts to the influence of UV radiation with the formation of melanin. Human melanins are biopolymers synthesized by the melanocytes. The melanocytes are localized in the epidermis of the skin. They have dendritic branches, via which they are connected to the keratinocytes. In the melanocytes, the melanin is formed and then transferred to the adjacent keratinocytes with the help of the melanosomes. In order to trigger the formation of melanin, the keratinocytes send out paracrine signals. Thus, e.g. in the keratinocytes, as a consequence of UV-A and UV-B irradiation, NO is formed which triggers the formation of melanin in the melanocytes. The melanocytes react to NO with increased cell growth, form dendrites to an increased extent and increase the melanogenesis. Regulation of melanogenesis in the melanocytes is moreover subject to control by the hormone alpha-MSH.

Local hyperpigmentations or even natural skin pigmentation are often perceived as cosmetic flaws. Various strategies have therefore been developed to reduce the pigmentation of the skin.

One of the most often used skin and hair lighteners is hydroquinone or the hydroquinone glycoside of arbutin. However, these compounds have a cytotoxic effect on melanocytes and have an irritative effect on the skin. Another option is the inhibition of the synthesis of melanin by inhibiting the pacemaker enzyme tyrosinase. For this, the substances kojic acid and derivatives of kojic acid such as e.g. kojic acid dipalmitate kojic acid, azelaic acid, oxyresveratrol, linolenic acid, vitamin C and derivatives of ascorbic acid such as, for example, ascorbyl phosphate or ascorbyl palmitate, inter alia, are used. However, these substances either have a high sensitizing potential, cause contact allergies, exhibit inadequate chemical stability in cosmetic formulations or have only an unsatisfactory effect on the skin.

Moreover, strategies are also known which prevent the transfer of the melanin from the melanocytes into the surrounding keratinocytes. Thus, protease inhibitors are described which inhibit the PAR2 receptor on the surface of the keratinocytes and as a result reduce the transfer of the melanin. Hydrolysates from soya beans and niacinamide are said to reduce pigmentation in this way.

Similarly, a skin-lightening effect is acknowledged for various plant extracts such as, for example, licorice extract, mulberry tree extract, or bearberry. Here, there is the problem of inadequate standardization of the extracts for a consistent effectiveness on the skin. Increased renewal of the skin is also described for lightening the skin. For this procedure, alpha-hydroxy acids such as lactic acid and glycolic acid, inter alia, are used. By means of this treatment, the uppermost skin layers are corroded away and the melanin-containing corneocytes are abraded. A disadvantage of this method is frequent irritation of the skin.

There is thus furthermore an increasing need for new, further and improved active ingredients for the treatment of hyperpigmentation, but also for the purely cosmetic lightening of relatively large areas of pigmented skin that are entirely appropriate to the individual skin type per se.

The peptide PKEK is described in the patent application WO/2008/085494 and in WO/2009/068351. It has immunomodulatory properties and also anti-aging efficacy.

It was an object of the invention to provide a care active ingredient which has a skin-lightening effect, which is well tolerated and can be formulated easily.

### DESCRIPTION OF THE INVENTION

Surprisingly, the object is achieved through the use of the tetrapeptide PKEK (Seq. ID No. 1) to combat undesired pigmentation of the skin.

The present invention therefore provides the use of the tetrapeptide PKEK or one of its derivatives for lightening the color of human skin, for bleaching pigment spots and/or for evening out irregularities in skin coloration.

The invention further provides the use of the tetrapeptide PKEK or one of its derivatives for producing a formulation, and also these specific formulations themselves.

It is an advantage of the present invention that the tetrapeptide itself has further properties which are advantageous in connection with skin lightening, such as, for example, the ability to tighten skin and to smooth skin wrinkles, as well as to alleviate inflammations.

Unless stated otherwise, all stated percentages (%) are percentages by mass.

A derivative of the tetrapeptide is to be understood as meaning in particular acyl derivatives; for the acyl derivatization of the tetrapeptide used according to the invention, as a result of amide bonding, an alkylic lipophilic chain or an arylic radical or alkyloxic or aryloxic or alkylaryloxic variants thereof can be attached to the N-terminal end of the oligopeptide and/or to the C-terminal end, as a result of ester bonding an alkyl alcohol or as a result of amide bonding an NH₂ group or one such N-alkyl-substituted group.

According to the invention an acyl group is preferably arranged on the N-terminal end of the amino acid sequence. This can optionally carry branched or straight-chain, long- or short-chain, saturated or unsaturated radicals, and be unsubstituted or substituted with one or more hydroxyl, amino, acylamino, sulfate or sulfide groups. Such N-acylic derivatives can be produced, for example, with acetic acid, biotinic acid, caprylic acid, capric acid, lauric acid, myristic acid, octanoic acid, palmitic acid, stearic acid, behenic acid, linoleic acid, linolenic acid, lipoic acid, oleic acid, isostearic acid, elaidic acid, 2-ethylhexanoic acid, coconut oil fatty acid, talc fatty acid, hydrogenated talc fatty acid, palm kernel oil fatty acid, lanolin fatty acid or mixtures thereof.

Preferred acyl groups include substituted or unsubstituted acetyl, palmitoyl, hexanoyl, myristyl, biotinyl and octanoyl groups.

The use according to the invention of the tetrapeptide PKEK or one of its derivatives for lightening the color of human skin, for bleaching pigment spots and/or for evening out irregularities in skin coloration belongs in particular to the cosmetic, non-therapeutic field.

The tetrapeptide PKEK or one of its derivatives can advantageously be used for producing a formulation for lightening the color of human skin, for bleaching pigment spots and/or for evening out irregularities in skin coloration.

A formulation for lightening the color of human skin, for bleaching pigment spots and/or for evening out irregularities in skin coloration are those, preferably cosmetic, formulations which are obviously formulated for such a purpose. This can be ascertained in particular from the fact that other active ingredients for lightening the skin are present. These may be in particular kojic acid, kojic acid derivatives, niacin/niacinamide, alpha-hydroxycarboxylic acids such as lactic acid, arbutin, arbutin derivatives, ascorbic acid, ascorbic acid derivatives such as sodium ascorbyl phosphate, magnesium ascorbyl phosphate and ascorbyl glucoside, hydroquinone, hydroquinone derivatives, glabridin in licorice, oleanoic acid, sulfur-containing molecules such as e.g. glutathione or cysteine or other synthetic or natural active ingredients for lightening the skin.

The invention further provides the use of the tetrapeptide PKEK or one of its derivatives for producing a sunscreen formulation.

The reason for this is that a sunscreen formulation has obviously been provided in order to likewise counteract coloration of the skin, only in this case it is prophylactic.

The obvious preparation of a sunscreen formulation can be ascertained in particular from the fact that substances that absorb UV radiation are present. Examples of such substances are listed below.

Consequently, the present invention also further provides a formulation, preferably a cosmetic formulation, comprising

a) an effective amount of the tetrapeptide PKEK or one of its derivatives
b) a safe and effective amount of at least one additional active ingredient selected from the group consisting of the active ingredients for bleaching the skin, depigmentation, sun protection and blocking UV rays and optionally
c) a dermatologically acceptable carrier.

The formulation “dermatologically acceptable carrier” means here that the carrier is suitable for topical application on the horny tissue, has good esthetic properties, is compatible with the ingredients of the present invention and any desired other components and does not lead to unfavorable safety and toxicity concerns.

The carrier can be present in many different forms. For example, emulsion carriers including oil-in-water, water-in-oil, water-in-oil-in-water and oil-in-water-in-silicone emulsions can be used here.

In connection with the formulations according to the invention, preferred active ingredients for the skin bleaching and the depigmentation are kojic acid, kojic acid dipalmitate, niacin/niacinamide, alpha-hydroxycarboxylic acids such as lactic acid, arbutin, ascorbic acid, ascorbic acid derivatives such as sodium ascorbyl phosphate, magnesium ascorbyl phosphate and ascorbyl glucoside ascorbyl palmitate, sodium ascorbyl phosphate, magnesium ascorbyl phosphate and ascorbyl glucoside, hydroquinone, glabridin in licorice, oleanoic acid, glutathione, cysteine, azelaic acid, oxyresveratrol, linolenic acid, dicarboxylic acids such as dioic acid.

These are present particularly when the formulation according to the invention is a formulation for lightening the color of human skin, for bleaching pigment spots.

In connection with the formulations according to the invention, preferred active ingredients for sun protection and blocking UV rays are selected from the group consisting of:

3-benzylidenecamphor, 3-(4-methylbenzylidene)camphor, 2-ethylhexyl 4-(dimethylamino)benzoate, 2-ethylhexyl 4-(dimethylamino)benzoate, amyl 4-(dimethylamino)benzoate, esters of cinnamic acid, esters of salicylic acid, benzophenone, 2-hydroxy-4-methoxybenzophenone, 2-hydroxy-4-methoxy-4″-methylbenzophenone, 2,2″-dihydroxy-4-methoxybenzophenone, esters of benzalmalonic acid, triazines, 2,4,6-trianilino(p-carbo-2″-ethyl-1″-hexyloxy)-1,3,5-triazine, octyltriazone, propane-1,3-diones, 1-(4-tert-butylphenyl)-3-(4″-methoxyphenyl)propane-1,3-dione, 2-phenylbenzimidazole-5-sulfonic acid, sulfonic acid derivatives of benzophenone, sulfonic acid derivatives of 3-benzylidenecamphor, derivatives of benzoylmethane, finely dispersed metal oxides and salts such as titanium dioxide or zinc oxide, 2,2″-methylenebis-{6-(2H-benzotriazol-2-yl)-4-(1,1,3,3-tetramethylbutyl)phenol.

These are present particularly when the formulation according to the invention is a sunscreen formulation.

According to the invention, particular preference is given to a formulation which is a formulation for evening out irregularities in skin coloration. This is characterized in particular in that it comprises an additional component d), which comprises self-tanning agents, preferably consists of these.

According to the invention, suitable self-tanning agents in this connection are selected from the group consisting of dihydroxyacetone and erythrulose.

The formulations according to the invention comprise from 0.00001 percent by mass to 1 percent by mass, preferably 0.00005 percent by mass to 0.5 percent by mass, particularly preferably 0.0001 percent by mass to 0.1 percent by mass of tetrapeptide or tetrapeptide derivative, based on the total mass of the formulation.

The formulation according to the invention can comprise e.g. at least one additional component selected from the group of
emollients,
emulsions,
thickeners/viscosity regulators/stabilizers,
antioxidants,
hydrotropes (or polyols),
solids and fillers,
pearlescent additives,
deodorant and antiperspirant active ingredients,
insect repellents,
self-tanning agents,
preservatives,
conditioners,
perfumes,
dyes,
cosmetic active ingredients,
care additives,
superfatting agents,
solvents.

Substances which can be used as exemplary representatives of the individual groups are known to the person skilled in the art and can be found, for example, in the German application DE 102008001788.4. This patent application is hereby incorporated by reference and thus forms part of the disclosure.

As regards further optional components and the amounts of these components used, reference is made expressly to the relevant handbooks known to the person skilled in the art, e.g. K. Schrader, “Grundlagen and Rezepturen der Kosmetika” [“Principles and Formulations of Cosmetics”], 2nd edition, page 329 to 341, Hüthig Buch Verlag Heidelberg.

The amounts of the particular additives are governed by the intended use.

Typical guide formulations for the particular applications are known prior art and are contained, for example, in the brochures from the manufacturers of the particular basic materials and active ingredients. These existing formulations can generally be adopted unchanged. If necessary, however, the desired modifications can be undertaken by simple experiments without complication for the purposes of adaptation and optimization.

The formulations according to the invention can, as described above for the tetrapeptide itself, be used advantageously for lightening the color of human skin, for bleaching pigment spots and/or for evening out irregularities in skin coloration.

The invention further provides a method for lightening the color of human skin, for bleaching pigment spots and/or for evening out irregularities in skin coloration, involving the process steps

A) provision of a formulation according to the invention
B) application of the formulation according to the invention to the skin at least once per day in an effective amount.

In the examples listed below, the present invention is described by way of example without intending to limit the invention, the scope of application of which arises from the entire description and the claims, to the embodiments specified in the examples.

The following figures form part of the examples:

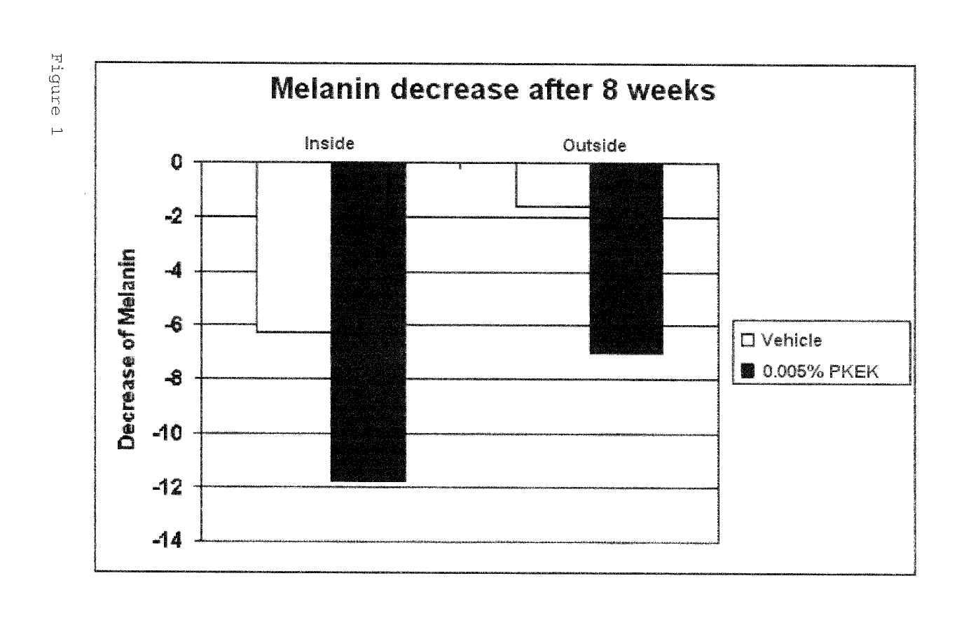

FIG. 1: melanin decrease in the panel test

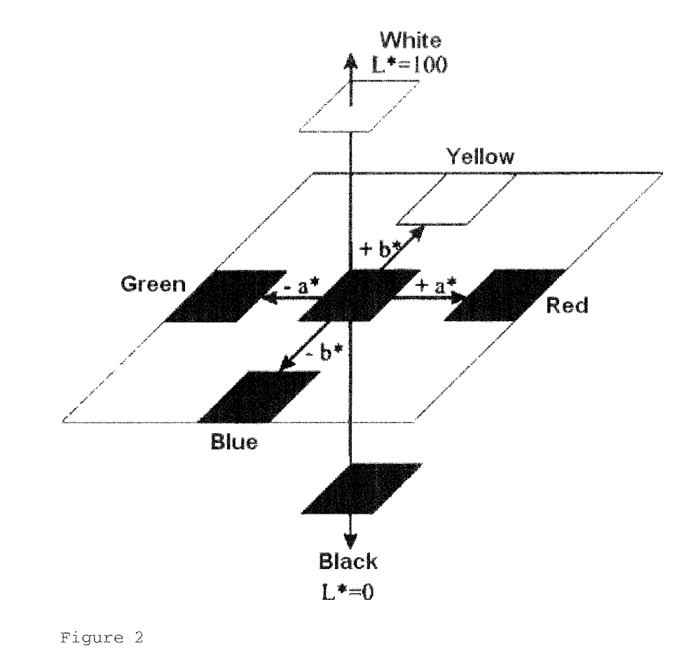

FIG. 2: color space L*a*b*

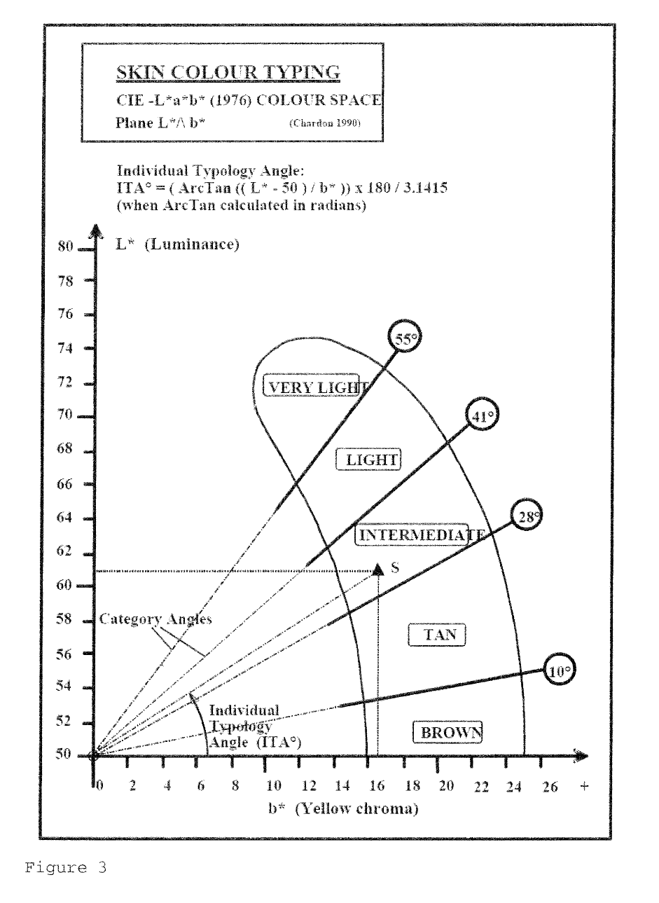

FIG. 3: typing of the different skin shades

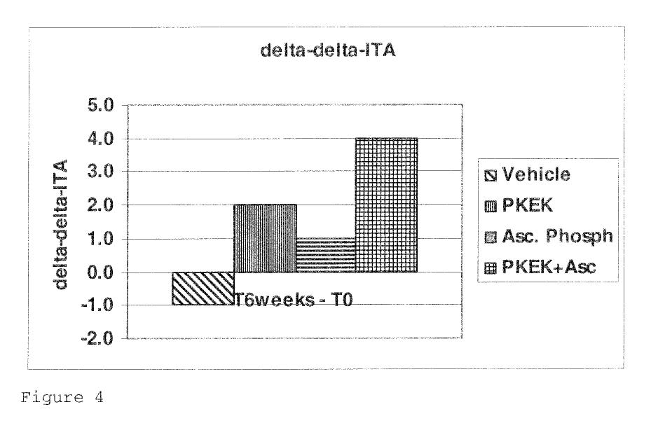

FIG. 4: ΔΔITA after 6-week application of the test formulations

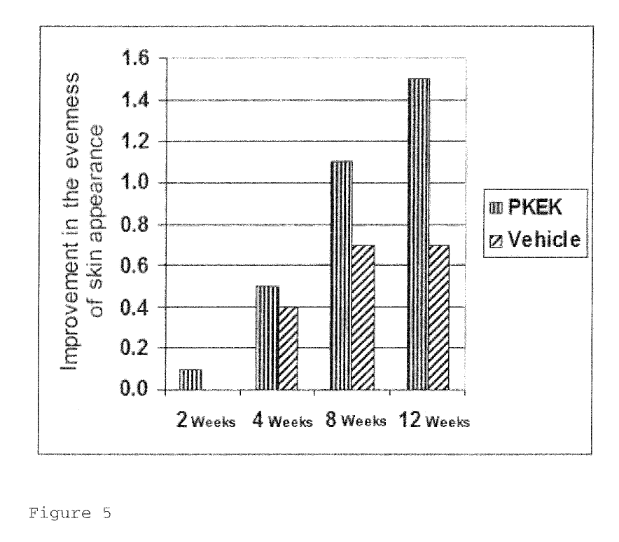

FIG. 5: improvement in the evenness of the skin appearance (** p<0.01 compared to PKEK/vehicle).

### EXAMPLES

### Example 1

### In Vivo Lightening (2-Month Study)

20 subjects wore a test formulation without peptide or with 0.005% PKEK over a period of 8 weeks on one forearm in each case. Prior to the start of the study, and also after 8 weeks, a Mexameter probe (Courage & Khazaka, Cologne) was used to measure the melanin concentration both on the inside and also on the outside of the forearm.

The measurement of the melanin concentration in the skin is based on the principle of absorption/reflection. The

Mexameter probe emits light of a specific wavelength which is in agreement with the absorption maxima of the melanin in the skin. The light reflected by the skin is measured and placed relative to the emitted amount of light. The resulting measurement values are given as index numbers.

### Test Formulation:

| | 0.005% H1410 | Vehicle |
|---|---|---|
| Polyglyceryl-3 Methylglucose | 3.0% | 3.0% |
| Distearate | | |
| Glyceryl Stearate | 2.0% | 2.0% |
| Stearyl Alcohol | 1.0% | 1.0% |
| PPG-3 Myristyl Ether | 9.5% | 9.5% |
| Caprylic/Capric Triglyceride | 9.5% | 9.5% |
| PKEK | 0.005% | — |
| Water | 74.195% | 74.2% |
| Microcare MEM | 0.8% | 0.8% |
| (methylisothiazolinone, methyl | | |
| paraben, ethyl paraben) | | |
| Perfume | q.s. | q.s. |

FIG. 1 shows the difference in the melanin values after 8 weeks compared to the starting value. Both on the inside and also on the outside of the forearm, a significantly greater decrease in skin brownness is established compared to the vehicle.

### Example 2

### Example Formulations

Example formulations are described below. The stated percentages are percentages by mass and refer to the total mass of the example formulation. To produce the formulations, customary formulation processes known to the person skilled in the art were used.

### O/W Formulation

| | |
|---|---|
| Phase A | |
| Polyglyceryl-3 Methylglucose Distearate | 3.0% |
| Glyceryl Stearate | 2.0% |
| Stearyl Alcohol | 1.0% |
| Decyl Cocoate | 10.0% |
| Cetearyl Ethylhexanoate | 9.0% |
| Phase B | |
| Glycerin | 3.0% |
| PKEK | 0.001% |
| Water | 71.999% |
| Phase Z | |
| Preservative, perfume | q.s. |

### W/O Formulation

| | |
|---|---|
| Phase A | |
| Polyglyceryl-4 Isostearate | 1.5% |
| Cetyl PEG/PPG-10/1 Dimethicone | 1.0% |
| Ethylhexyl Palmitate | 11.0% |
| Decyl Oleate | 10.5% |
| Hydrogenated Castor Oil | 0.8% |
| Microcrystalline Wax | 1.2% |
| Phase B | |
| Glycerin | 3.0% |
| Magnesium Sulfate Heptahydrate | 0.6% |
| PKEK | 0.002% |
| Water | 70.398% |
| Phase Z | |
| Preservative, perfume | q.s. |

### Shower Gel

| | |
|---|---|
| Water | 50.248% |
| PKEK | 0.002% |
| Polyquaternium-10 | 0.3% |
| Sodium Laureth Sulfate, 28% | 36.0% |
| Disodium Cocoamphodiacetate, 39% | 6.0% |
| Cocamidopropylbetaine; Glyceryl Laurate | 5.0% |
| PEG-7 Glyceryl Cocoate | 1.6% |
| Sodium Chloride | 0.85% |
| Preservative, perfume | q.s. |

O/W Cream with Licorice Extract

| | |
|---|---|
| Phase A | |
| Stearyl Alcohol | 3.5% |
| Glyceryl Stearate | 1.5% |
| Cetearyl Ethylhexanoate | 7.8% |
| Caprylic/Capric Triglyceride | 10.0% |
| Macadamia ternifolia Nut Oil | 4.0% |
| Tocopheryl Acetate | 1.0% |
| Dimethicone | 0.2% |
| Phase B | |
| Cetearyl Glucoside | 1.0% |
| Sucrose Stearate | 2.0% |
| Glycerin | 3.0% |
| PKEK | 0.005% |
| Glycyrrhiza glabra (licorice) extract | 0.1% |
| Water | 64.895% |
| Phase C | |
| Carbomer | 0.2% |
| Cetearyl Ethylhexanoate | 0.8% |
| Phase D | |
| Sodium Hydroxide (10%) | q.s. |
| Phase Z | |
| Preservative, perfume | q.s. |

O/W Cream with Licorice Root Extract

| | |
|---|---|
| Phase A | |
| Stearyl Alcohol | 3.0% |
| Glyceryl Stearate | 1.0% |
| Stearic Acid | 1.0% |
| Caprylic/Capric Triglyceride | 9.0% |
| Decyl Cocoate | 4.5% |
| Avocado (Persea gratissima) Oil | 5.0% |
| Tocopheryl Acetate | 0.5% |
| Phase B | |
| Cetearyl Glucoside | 1.0% |
| Glycerin | 3.0% |
| Allantoin | 0.2% |
| Panthenol | 0.5% |
| PKEK | 0.002% |
| Glycyrrhiza glabra (licorice) root | 0.1% |
| extract | |
| Water | 69.198% |
| Phase C | |
| Sodium Lactate; Sodium PCA; Fructose; | 2.0% |
| Urea; Niacinamide; Inositol; Sodium | |
| Benzoate; Lactic Acid | |
| Phase Z | |
| Preservative, perfume | q.s. |

O/W Body Lotion with Kojic Acid

| | |
|---|---|
| Phase A | |
| Glyceryl Stearate Citrate | 1.5% |
| Cetearyl Alcohol | 1.0% |
| Caprylic/Capric Triglyceride | 7.0% |
| Mineral Oil | 5.5% |
| Phase B | |
| Glycerin | 5.0% |
| PKEK | 0.003% |
| Kojic acid | 0.2% |
| Water | 78.197% |
| Phase C | |
| Carbomer | 0.2% |
| Mineral Oil | 0.8% |
| Phase D | |
| Sodium Hydroxide (10%) | 0.6% |
| Phase Z | |
| Preservative, perfume | q.s. |

W/O Body Lotion with Arbutin

| | |
|---|---|
| Phase A | |
| Diisostearoyl Polyglyceryl-3 Dimer Dilinoleate | 3.0% |
| Hydrogenated Castor Oil | 0.2% |
| Microcrystalline Wax | 0.3% |
| Isocetyl Palmitate | 8.0% |
| Ethylhexyl Palmitate | 5.5% |
| Isopropyl Palmitate | 8.0% |
| Phase B | |
| Glycerin | 2.0% |
| Magnesium Sulfate Heptahydrate | 1.0% |
| PKEK | 0.001% |
| Arbutin | 2.0% |
| Water | 69.999% |
| Phase Z | |
| Preservative, perfume | q.s. |

O/W Body Butter with Hydroquinone

| | |
|---|---|
| Phase A | |
| Glyceryl Stearate; PEG-100 Stearate | 6.0% |
| Cetearyl Alcohol | 1.5% |
| Myristyl Myristate | 1.0% |
| Cetyl Ricinoleate | 1.0% |
| Cyclomethicone | 6.0% |
| Behenoxy Dimethicone | 1.0% |
| Soybean (Glycine soja) Oil | 7.0% |
| Butyrumspermum parkii (Shea Butter) | 7.0% |
| Lanolin Alcohol | 1.0% |
| Theobroma cacao (Cacao Butter) | 7.0% |
| Phase B | |
| Glycerin | 5.0% |
| EDTA | 0.1% |
| PKEK | 0.002% |
| Hydroquinone | 0.5% |
| Water | 55.898% |
| Phase Z | |
| Preservative, perfume | q.s. |

O/W Cream with Mulberry Extract

| | |
|---|---|
| Phase A | |
| Bis-PEG/PPG-16/16 PEG/PPG 16/16 | 1.5% |
| Dimethicone; Caprylic/Capric | |
| Triglyceride | |
| Ceteareth-25 | 1.0% |
| Glyceryl Stearate | 3.0% |
| Stearyl Alcohol | 2.0% |
| Stearic Acid | 1.0% |
| Isocetyl Palmitate | 5.5% |
| Ethylhexyl Palmitate | 6.0% |
| Phase B | |
| Glycerin | 2.0% |
| PKEK | 0.005% |
| Water | 76.495% |
| Phase C | |
| Carbomer | 0.1% |
| Isocetyl Palmitate | 0.4% |
| Phase D | |
| Sodium Hydroxide (10%) | q.s. |
| Phase E | |
| Butylene Glycol, Morus alba root | 1.0% |
| extract | |
| Phase Z | |
| Preservative, perfume | q.s. |

O/W Cream with Oxyresveratrol

| | |
|---|---|
| Phase A | |
| Polyglyceryl-3 Methylglucose Distearate | 3.0% |
| Glyceryl Stearate | 2.0% |
| Stearyl Alcohol | 1.0% |
| PPG-3 Myristyl Ether | 9.5% |
| Ethylhexyl Stearate | 9.5% |
| Phase B | |
| Glycerin | 3.0% |
| PKEK | 0.002% |
| Oxyresveratrol | 0.2% |
| Water | 71.798% |
| Phase Z | |
| Preservative, perfume | |

O/W Cream with Octadecenedioic Acid

| | |
|---|---|
| Phase A | |
| Ceteareth-25 | 2.0% |
| Stearyl Alcohol | 2.5% |
| Glyceryl Stearate | 1.5% |
| Stearic Acid | 1.0% |
| Ethylhexyl Stearate | 10.0% |
| Mineral Oil | 8.0% |
| Octadecenedioic Acid | 1.0% |
| Phase B | |
| Water | 69.999% |
| PKEK | 0.001% |
| Phase C | |
| Betaine | 2.0% |
| Water | 2.0% |
| Phase Z | |
| Preservative, perfume | q.s. |

W/O Cream with Magnesium Ascorbyl Phosphate

| | |
|---|---|
| Phase A | |
| Cetyl PEG/PPG-10/1 Dimethicone | 2.0% |
| Polyglyceryl-4 Isostearate | 1.0% |
| Hydrogenated Castor Oil | 0.8% |
| Microcrystalline Wax | 1.2% |
| Isohexadecane | 9.5% |
| Caprylic/Capric Triglyceride | 6.5% |
| Cetearyl Ethylhexanoate | 4.0% |
| Phase B | |
| Creatine | 0.5% |
| PKEK | 0.003% |
| Magnesium Ascorbyl Phosphate | 1.5% |
| Sodium Chloride | 0.5% |
| Water | 72.497% |
| Phase Z | |
| Preservative, perfume | q.s. |

O/W Body Lotion with Sodium Ascorbyl Phosphate

| | |
|---|---|
| Phase A | |
| Glyceryl Stearate SE | 4.0% |
| Stearic Acid | 0.5% |
| Myristyl Alcohol | 0.5% |
| Mineral Oil | 4.6% |
| Ethylhexyl Stearate | 5.0% |
| Phase B | |
| Glycerin | 3.0% |
| PKEK | 0.001% |
| Water | 70.399 |
| Phase C | |
| Carbomer | 0.1% |
| Mineral Oil | 0.4% |
| Phase D | |
| Sodium Ascorbyl Phosphate | 1.5% |
| Water | 10.0% |
| Phase E | |
| Sodium Hydroxide (10%) | q.s. |
| Phase Z | |
| Preservative, perfume | q.s. |

Cream Bath with Niacinamide

| | |
|---|---|
| Almond (Prunus dulcis oil) | 0.3% |
| Perfume | 0.5% |
| PEG-20 Glyceryl Oleate | 2.0% |
| Sodium Laureth Sulfate, 28% | 40.7% |
| Quaternium-80 | 1.0% |
| Water | 37.499% |
| PKEK | 0.001% |
| Niacinamide | 0.5% |
| Lauryl Glucoside, 50% | 6.35% |
| Cocamidopropyl Betaine, 37.5% | 11.65% |
| PEG-18 Glyceryl Oleate/Cocoate | 1.5% |
| Glycol Distearate; Steareth-4 | 3.0% |

### O/W Sunscreen Lotion

| | |
|---|---|
| Phase A | |
| Polyglyceryl-3 Methylglucose Distearate | 2.5% |
| C12-15 Alkyl Benzoate | 4.0% |
| Decyl Cocoate | 2.5% |
| Isopropyl Palmitate | 1.6% |
| Tocopheryl Acetate | 0.5% |
| Ethylhexyl Methoxycinnamate | 5.0% |
| 4-Methylbenzylidene Camphor | 3.0% |
| Butyl Methoxydibenzoylmethane | 2.0% |
| Phase B | |
| Glycerin | 2.0% |
| EDTA | 0.1% |
| PKEK | 0.003% |
| Water | 75.897% |
| Phase C | |
| Carbomer | 0.05% |
| Acrylates/C10-30 Alkyl Acrylates Crosspolymer | 0.05% |
| Isopropyl Palmitate | 0.4% |
| Phase D | |
| Sodium Hydroxide (10%) | 0.4% |
| Phase Z | |
| Preservative, perfume | q.s. |

### W/O Sunscreen Cream

| | |
|---|---|
| Phase A | |
| Cetyl PEG/PPG-10/1Dimethicone | 2.5% |
| Diethylhexyl Carbonate | 7.4% |
| C12-15 Alkyl Benzoate | 2.0% |
| Cera Alba | 0.5% |
| Hydrogenated Castor Oil | 0.5% |
| Tocopheryl Acetate | 0.6% |
| Ethylhexyl Salicylate | 3.0% |
| Ethylhexyl Methoxycinnamate | 7.5% |
| Benzophenone-3 | 5.0% |
| Phase B | |
| Sodium Carboxymethyl Beta-Glucan | 0.2% |
| Glycerin | 1.0% |
| Allantoin | 0.1% |
| PKEK | 0.002% |
| Sodium Chloride | 0.5% |
| Water | 69.198% |
| Phase Z | |
| Preservative, perfume | q.s. |

### Example 3

### Reduction of Age Spots on the Face

Age spots (Latin: Lentigines seniles, Lentigines solares) are pigment disorders in the skin. They are produced by increased, chronic exposure to ultraviolet radiation, e.g. sunlight. This results in local, sharply delimited pale brown spot formations (“maculae”) with increase in the melanin-producing melanocytes, predominantly in the area of the backs of the hands, forearms and facial skin.

In order to quantify such age spots, color measurements of the skin are carried out. For this purpose, chromameters are used. The chromameter measures the color values L*, a* and b*. These describe a three-dimensional color space, with the help of which every perceivable color can be described, cf. FIG. 2.

On the a*-axis the opposite colors green and red face each other, and on the b*-axis the opposite colors blue and yellow face each other. The axis L* gives the lightness.

The end points are black (L=0) and white (L=100). In order to be able to classify the skin shade, the parameters L* and b* are required. The skin shade ITA° is given in degrees and calculated using the following formula:

ITA°=[arctan((L*−50)/b*)]*180/3.14159

The skin shade is classified in this connection as follows, cf. FIG. 3:

| | |
|---|---|
| ITA° > 55° | very light |
| 55° > ITA° > 41° | light |
| 41° > ITA° > 28° | intermediate |
| 28° > ITA° > 10° | tanned. |

See also in this regard: COLIPA Guideline: Guideline for the colometric determination of skin colour typing and prediction of the minimal erythemal dose (MED) without UV exposure.

By means of the following study, the aim is to investigate whether the tetrapeptide PKEK is able to visibly reduce age spots.

The study was carried out as a half-side test. 40 women aged between 30 and 70 years were selected who had visible age spots on the face. They were each given 2 test formulations, one for the right half of the face and the other for the left half of the face. The test formulations had to be applied twice daily over a period of six weeks.

| | | Asc. | PKEK + | |
|---|---|---|---|---|
| | PKEK | Phos. | Asc. Phos. | Vehicle |
| Polyglyceryl-3 | 3.0 | 3.0 | 3.0 | 3.0 |
| Methylglucose | | | | |
| Distearate | | | | |
| Stearyl Alcohol | 1.0 | 1.0 | 1.0 | 1.0 |
| Glyceryl Stearate | 2.0 | 2.0 | 2.0 | 2.0 |
| C12-15 | 9.5 | 9.5 | 9.5 | 9.5 |
| Alkylbenzoate | | | | |
| Caprylic/Capric | 9.5 | 9.5 | 9.5 | 9.5 |
| Triglyceride | | | | |
| Water | 61.496 | 59.7 | 59.696 | 61.5 |
| Sodium ascorbyl | | 1.5 | 1.5 | |
| phosphate | | | | |
| Sodium Bisulfite | | 0.3 | 0.3 | |
| (39%) | | | | |
| Urea | 2.5 | 2.5 | 2.5 | 2.5 |
| PKEK | 0.004 | | 0.004 | |
| Water | 10.0 | 10.0 | 10.0 | 10.0 |
| Microcare MEM | 0.8 | 0.8 | 0.8 | 0.8 |
| (preserver) | | | | |
| Perfume | 0.2 | 0.2 | 0.2 | 0.2 |
| pH value | >=7 | >=7 | >=7 | >=7 |

Test formulation (data in % by weight)

Prior to the start of the study and also after 6 weeks, the color of the skin directly on the age spot was measured as well as in the adjoining area. This was performed using a CR 400 chromameter from Minolta. The skin shade ITA° was calculated both for the age spot (ITA°_{A}) and also the adjoining area (ITA°_{U}) and the difference was calculated:

ΔITA°=ITA°_{U}−ITA°_{A}

The greater ΔITA°, the more visible the age spot. The difference of ΔITA° before the start of application of the test formulations and also after 6 weeks was then calculated (AΔITA):

ΔΔITA=ΔITA°ₛₜₐᵣₜ−ΔITA°_{6 we}

A visible reduction of the age spots is present when:

ΔITA°ₛₜₐᵣₜ>ΔITA°_{6 we}

[symbol]

ΔITA°ₛₜₐᵣₜ−ΔITA°_{6 we}>0

[symbol]

ΔΔITA>0

FIG. 4 shows the values for AΔITA after application of the test formulations for 6 weeks.

Both PKEK on its own and also sodium ascorbyl phosphate led to a visible reduction of the age spots. In this connection, PKEK even led to a greater reduction than sodium ascorbyl phosphate. This can be attributed to the fact that no active ingredient was present here which could compensate for negative effect on the skin, e.g. caused by UV radiation. By combining PKEK with sodium ascorbyl phosphate, the effectiveness of the cosmetic formulation was able to be increased synergistically.

### Example 4

### Improvement in the Skin Appearance in Dark-Skinned People

For the following study, dark-skinned women (Fitzpatrick type VI-V) were used. The Fitzpatrick scale serves to organize the different skin types:

| Skin | | |
|---|---|---|
| type | Characterization | Sensitivity to sun |
| I | Celtic type, very | Will not tan, very frequent |
| | light skin color, | sunburn, intrinsic |
| | reddish or pale | protection time |
| | blonde hair, blue, | <10 minutes. |
| | green or pale gray | |
| | eyes, freckles | |
| II | Nordic type, light | Slow, minimal tanning, |
| | skin color, blonde or | often sunburn, intrinsic |
| | pale brown hair, | protection time: 10-20 |
| | blue, gray or green | minutes. |
| | eyes, often freckles | |
| III | Mixed type, average | Slow, but developing tan, |
| | skin color, dark | sometimes sunburn, |
| | brown or pale brown | intrinsic protection time: |
| | hair, brown (blue, | 20-30 minutes |
| | green or gray) eyes, | |
| | rarely freckles | |
| IV | Mediterranean type, | Rapid tanning, rarely |
| | brownish or olive- | sunburn, intrinsic |
| | colored skin even in | protection time: |
| | the untanned state, | >30 minutes. |
| | brown eyes, brown or | |
| | black hair, no | |
| | freckles | |
| V | Dark skin type, dark | Rapid tanning, hardly any |
| | skin even in the | sunburn, intrinsic |
| | untanned state, dark | protection time: |
| | eyes, black hair, no | >60 minutes |
| | freckles | |
| VI | Black skin type, dark | Virtually never sunburn, |
| | brown to black skin | intrinsic protection time: |
| | even in the untanned | >90 minutes |
| | state, black eyes, | |
| | black hair, no | |
| | freckles | |

Compare also http://dermatology.about.com/od/cosmeticprocedure/a/fitzpatrick.htm

In each case, 25 female subjects were given a face cream which comprised either the tetrapeptide PKEK or no active ingredient (vehicle). They had to apply this formulation twice daily over the entire face for a period of 12 weeks. Prior to the start of the study and also after 2, 4, 8 and 12 weeks, the evenness of the skin appearance was assessed visually by an expert.

A five-part scale was used for assessing the skin appearance:

5=no contrast, very even skin appearance
4=slight contrast, slightly irregular skin appearance
3=moderate contrast, irregular skin appearance
2=high contrast, highly irregular skin appearance
1=very high contrast, very highly irregular skin appearance

The table below gives the composition of the test formulation:

| | PKEK | Vehicle |
|---|---|---|
| Polyglyceryl-3 Methylglucose | 3.0% | 3.0% |
| Distearate | | |
| Glyceryl Stearate | 2.0% | 2.0% |
| Stearyl Alcohol | 1.0% | 1.0% |
| PPG-3 Myristyl Ether | 9.5% | 9.5% |
| C12-15 Alkyl Benzoate | 9.5% | 9.5% |
| Water | 74.196% | 74.2% |
| PKEK | 0.004% | — |
| Microcare MEM (preserver) | 0.8% | 0.8% |
| pH (lactic acid 10%) | 6.0 ± 0.5 | 6.0 ± 0.5 |

Test Formulation for the In Vivo Study on Dark Skin

FIG. 5 shows that within the first 8 weeks the skin appearance of the subjects visibly improved with both test formulations, with the improvement by the tetrapeptide PKEK being increasingly greater over the course of the time than with the vehicle. After 12 weeks, with the vehicle no further improvement takes place, whereas PKEK significantly improves the skin appearance yet further.

### SEQUENCE LISTING

### <110> Evonik Goldschmidt GmbH

<120> Tetrapeptides for lightening the skin
<130>200900345
<160>1
<170> PatentIn version 3.4
<210>1
<211>4

### <212> PRT

### <213> Artificial

<220>

### <223> Artificial Peptide

<400>1

### Pro Lys Glu Lys

1

1. A method for treating human skin having at least one undesired skin pigmentation, said method comprising applying an effective amount of a tetrapeptide PKEK or a derivative thereof to said human skin at least once per day to reduce or even eliminate said at least one undesired skin pigmentation.

2. The method as claimed in claim 1, wherein said tetrapeptide PKEK or a derivative thereof is applied as one ingredient of a formulation, said formulation further comprises at least one additional active ingredient selected from the group consisting of active ingredients for bleaching the skin, depigmentation, sun protection and blocking UV rays.

3. The method as claimed in claim 2, wherein said formulation further comprises at least one dermatology acceptable carrier.

4. A formulation comprising:
a) an effective amount of tetrapeptide PKEK or a derivative; and
b) a safe and effective amount of at least one additional active ingredient selected from the group consisting of active ingredients for bleaching the skin, depigmentation, sun protection and blocking UV rays.

5. The formulation as claimed in claim 4, wherein said active ingredient for bleaching the skin or the active ingredient for the depigmentation is selected from the group consisting of:
kojic acid and its esters, niacin/niacinamides, alpha-hydroxycarboxylic acids, such as lactic acid, arbutin, ascorbic acid and its esters, hydroquinone, glabridin in licorice, oxyresveratrol and its derivatives, linolenic acid and its esters.

6. The formulation as claimed in claim 4, wherein the active ingredient for the sun protection or the active ingredient for blocking UV rays is selected from the group consisting of:
3-benzylidenecamphor, 3-(4-methylbenzylidene)camphor, 2-ethylhexyl 4-(dimethylamino)benzoate, amyl 4-(dimethylamino)benzoate, esters of cinnamic acid, esters of salicylic acid, benzophenone, 2-hydroxy-4-methoxybenzophenone, 2-hydroxy-4-methoxy-4′-methylbenzophenone, 2,2′-dihydroxy-4-methoxybenzophenone, esters of benzalmalonic acid, triazines, 2,4,6-trianilino(p-carbo-2′-ethyl-1′-hexyloxy)-1,3,5-triazine, octyltriazone, propane-, 3-diones, 1-(4-tert-butylphenyl)-3-(4′-methoxyphenyl)propane-1,3-dione, 2-phenylbenzimidazole-5-sulfonic acid, sulfonic acid derivatives of benzophenone, sulfonic acid derivatives of 3-benzylidenecamphor, derivatives of benzoylmethane, finely dispersed metal oxides or salts, and 2,2″-methylenebis-{6-(2H-benzotriazol-2-yl)-4-(1,1,3,3-tetramethylbutyl)phenol.

7. The cosmetic formulation as claimed in claim 4, further comprising a self-tanning agent.

8. The formulation as claimed in claim 4, further comprising at least one dermatologically acceptable carrier.

9. The formulation as claimed in claim 4, wherein said effective amount of said tetrapeptide PKEK or said derivative thereof is from 0.00001 percent by mass to 1 percent by mass of the total mass of the formulation.

10. The formulation as claimed in claim 4, wherein said effective amount of said tetrapeptide PKEK or said derivative thereof is from 0.00005 percent by mass to 0.5 percent by mass of the total mass of the formulation.

11. The formulation as claimed in claim 4, wherein said effective amount of said tetrapeptide PKEK or said derivative thereof is from 0.0001 percent by mass to 0.1 percent by mass of the total mass of the formulation.

12. The formulation as claimed in claim 8, wherein said dermatologically acceptable carrier is an emulsion.

13. The formulation as claimed in claim 12, wherein said emulsion is selected from the group consisting of an oil-in-water emulsion, a water-in-oil emulsion, a water-in-oil-in-water emulsion, and an oil-in-water-in-silicone emulsion.

14. The formulation as claimed in claim 7, wherein said self-tanning agent is selected from the group consisting of dihydroxyacetone and erthrulose.

15. The formulation as claimed in claim 4, further comprising at least one additional component selected from the group consisting of emollients, emulsions, thickeners/viscosity regulators/stabilizers, antioxidants, hydrotropes (or polyols), solids and fillers, pearlescent additives, deodorant and antiperspirant active ingredients, insect repellents, self-tanning agents, preservatives, conditioners, perfumes, dyes, cosmetic active ingredients, care additives, superfatting agents, and solvents.
